(19) United States
(12) Patent Application Publication
(10) Pub. No.: US 2025/0269180 A1
(43) Pub. Date: Aug. 28, 2025

(54) DEVICES AND METHODS FOR TREATING TESTICULAR PAIN

(71) Applicant: PathRight Medical, Inc., Plymouth, MN (US)

(72) Inventor: Landon Trost, Orem, UT (US)

(21) Appl. No.: 18/590,400

(22) Filed: Feb. 28, 2024

(51) Int. Cl. A61N 1/36; A61N 1/04

(52) U.S. Cl. CPC A61N 1/36021; A61N 1/0492; A61N 1/36034

(57) ABSTRACT

The present disclosure relates to a method of treating testicular pain. The method includes externally coupling an electrical energy source to a pelvic region of a subject, coupling a vibration mechanism to the pelvic region of the subject, delivering electrical energy to the pelvic region, and applying vibrations to the pelvic region.

### TECHNICAL FIELD

The present disclosure relates to devices and methods for treating testicular pain, and in particular, to devices and methods that deliver electrical energy and vibration to a pelvic region to treat testicular pain.

### BACKGROUND

Testicular pain may be caused by a variety of injuries or disorders in men. For example, iliohypogastric neuralgia, ilioinguinal neuralgia, genitofemoral neuralgia, chronic groin pain, chronic testicular pain (CTP), post vasectomy pain, hypogastric nerve injury, anterior extrusions, and other pain originating from the testicles, groin, or abdomen are common reasons for referral to a urological specialist. The testicular pain experienced by a subject may be unilateral or bilateral, constant or intermittent, spontaneous or exacerbated by physical activities and pressure. Pain may remain localized in the scrotum or radiate to throughout the pelvic region to the groin or perineum, or extend to the back or legs.

### SUMMARY

According to the present disclosure, devices and methods for non-invasively treating testicular pain using electrical and vibrational stimulation may be adapted to a wide variety of physiologies of subjects to treat pain.

In a first example aspect, a method of treating testicular pain may include coupling an electrical energy source to a pelvic region of the subject, coupling a vibration mechanism to the pelvic region of the subject, delivering electrical energy to the pelvic region, and applying vibrations to the pelvic region.

In a second example aspect, a method of treating testicular pain in a subject may include coupling a stimulation device to a pelvic region of the subject. The stimulation device may include an electrical contact configured for delivering electrical energy and a vibration mechanism configured for delivering vibrations. The method may include delivering, via the stimulation device, electrical energy to the subject. The method may include delivering, via the stimulation device, vibrations to the subject.

In a third example aspect, a wearable device for treating testicular pain may include a first stimulation contact configured for delivering an electrical pulse to a pelvic region of a subject, and a second stimulation contact configured for delivering vibrations to the pelvic region of the subject.

In accordance with any one of the first, second, and third example aspects, a method for treating testicular pain and a wearable stimulation device to treat testicular pain may include any one or more of the following forms.

In one form, coupling the electrical energy source may include externally engaging the electrical energy source with a scrotum of the subject.

In some forms, coupling the vibration mechanism may include engaging the vibration mechanism with a scrotum of the subject.

In other forms, coupling the electrical energy source and coupling the vibration mechanism may include connecting a stimulation device to the pelvic region of the subject.

In some forms, the stimulation device may include an electrode and the vibration mechanism.

In another form, coupling the stimulation device may include attaching a cuff at least partially around a scrotum of the subject.

In some forms, the cuff may include the electrode and the vibration mechanism.

In some forms, the electrode and the vibration mechanism may be arranged to engage the scrotum when the cuff is attached to the scrotum.

In yet another form, the method may include positioning the vibration mechanism in a first area of the pelvic region and positioning the electrode in a second area of the pelvic region.

In one example, coupling the electrical energy source may include adhering an electrode to the subject.

In some examples, delivering the electrical energy may include delivering an electrical pulse at a frequency in a range of approximately 0 Hz to approximately 150 Hz.

In other examples, applying vibrations may include applying vibrations having a frequency in a range of approximately 0 Hz to approximately 50 Hz.

In another example, applying vibrations and delivering the electrical energy may include delivering an electrical pulse and applying vibrations to a testicular cord, a cremasteric muscle, an inguinal canal, a genitofemoral nerve, an ilioinguinal nerve, and/or an iliohypogastric nerve of the subject.

In yet another example, coupling the stimulation device may include engaging one or more of the electrical contacts and the vibration mechanism to a scrotum of the subject.

In some examples, coupling the stimulation device may include positioning a second electrical contact of the stimulation device in an area of the pelvic region that is different than the scrotum of the subject.

In one aspect, coupling the stimulation device may include fastening a cuff of the stimulation device around a scrotum of the subject.

In another aspect, the electrical contact and the vibration mechanism may be integrated with the cuff of the stimulation device.

In another aspect, the method may include adjusting, via a controller, an intensity of a deliverable electrical energy and/or deliverable vibrations.

In some aspects, the controller may be operatively coupled to the electrical contact and to the vibration mechanism.

In yet another aspect, adjusting may include adjusting a stimulation setting using a personal electronic device.

In some aspects, the controller may be communicatively coupled to the personal electronic device.

In other aspects, a flexible band may be arranged for removably attaching the first stimulation contact and the second stimulation contact to a scrotum of the subject.

In some aspects, the first stimulation contact and second stimulation contact may be coupled to the flexible band.

In one form, a controller may be operatively coupled to the first stimulation contact and the second stimulation contact.

In some forms, the controller may include a processor, a memory communicatively coupled to the processor and storing executable instructions that, when executed by the processor, causes the processor to receive data transmitted by a remote communication device, send a signal to a generator coupled to the first stimulation contact, adjust an energy parameter of the electrical pulse generated by the generator, send a signal to a vibratory element of the second stimulation contact, and adjust an intensity parameter of vibrations generated by the vibratory element.

In one form, externally coupling an electrical energy source may include engaging the electrical energy source with a scrotum of the subject.

In some forms, externally coupling the electrical energy source may include coupling an electrical stimulation device to a scrotum of the subject.

In other forms, wherein the electrical energy source may include a first electrode and a second electrode of the electrical stimulation device.

In another form, coupling the electrical stimulation device may include attaching a cuff at least partially around the scrotum of the subject.

In some forms, the cuff may include an electrode positioned to engage the scrotum when the cuff is attached to the scrotum.

In yet another form, externally coupling the electrical energy source may include coupling an electrode of an electrical stimulation device to a scrotum of the subject.

In one example, externally coupling the electrical energy source may include coupling a second electrode to a different area of the pelvic region.

In some examples, externally coupling the electrical energy source may include adhering an electrode to a pelvic region adjacent to an inguinal canal and/or cremasteric muscle.

In other examples, delivering the electrical energy may include delivering an electrical pulse to the inguinal canal and/or cremasteric muscle of the subject.

In yet another example, delivering the electrical energy may include delivering an electrical pulse to a testicular cord, a cremasteric muscle, an inguinal canal, a genitofemoral nerve, an ilioinguinal nerve, and/or an iliohypogastric nerve of the subject.

In one aspect, the method may include adjusting a frequency of the electrical pulse being delivered.

In some aspects, the frequency may be in a range of approximately 0 Hz to approximately 150 Hz.

In some aspects, the method may include adjusting a pulse width of the electrical pulse being delivered.

In one aspect, the pulse width may be in a range of approximately 50 microseconds to approximately 200 microseconds.

In other aspects, externally coupling may include coupling a first electrode of an electrical stimulation device to a first area of the pelvic region and coupling a second electrode of the electrical stimulation device to a second area of the pelvic region.

In another aspect, coupling the stimulation device may include positioning a second contact in proximity to an inguinal canal of the subject.

In yet another aspect, coupling the stimulation device may include positioning a second contact of the stimulation device in an area different than the scrotum in a pelvic region of the subject.

In one form, the method may include positioning a cuff of the stimulation device around the scrotum of the subject.

In other forms, the contact may be integrated with the cuff of the stimulation device.

In some forms, delivering electrical energy may include delivering an electrical pulse to the subject.

In other forms, the method may include adjusting a setting of the electrical pulse.

In another form, adjusting the setting may include communicating with a controller using a remote communication device.

In some forms, the controller may be communicatively coupled to the electrical energy generator.

In yet another form, the method may include adjusting a frequency of the one or more electrical pulses being delivered.

In one example, the frequency may be in a range of approximately 0 Hz to approximately 150 Hz.

In one example, the method may include adjusting a pulse width of the electrical pulse being delivered.

In some examples, the pulse width may be in a range of approximately 50 microseconds to 200 microseconds.

In some examples, delivering the electrical energy may include delivering an electrical pulse to an inguinal canal of the subject.

In other examples, delivering the electrical energy may include delivering an electrical pulse to a genitofemoral nerve of the subject.

In yet another example, delivering the electrical energy may include delivering an electrical pulse to an ilioinguinal nerve of the subject.

In one aspect, delivering the electrical energy may include delivering an electrical pulse to an iliohypogastric nerve of the subject.

In some aspects, delivering the electrical energy may include delivering an electrical pulse to a cremasteric muscle of a testicular cord.

In other aspects, the device may include a garment defining a pocket sized to receive the generator.

In some aspects, the generator may be insertable in the pocket of the garment.

In another aspect, the stimulation contact may include an electrode configured for delivering an electrical pulse.

In yet another aspect, the device may include a flexible band arranged for removably attaching to a scrotum of a subject.

In one form, the electrical stimulation source may be coupled to the band.

In one form, the band may be arranged to form a circular cuff sized to fit around a scrotum of a subject.

In some forms, a controller may be operatively coupled to the generator.

In another form, the controller may include a processor and a memory communicatively coupled to the processor and storing executable instructions that, when executed by the processor, causes the processor to receive data transmitted by a remote communication device, and send a signal to the generator.

In other forms, the instructions may cause the processor to adjust an energy parameter of the energy generated by the generator.

In one form, applying vibrations may include applying vibrations to a testicular cord of the human subject.

In another form, applying vibrations may include applying vibrations to a cremasteric muscle of the human subject.

In some forms, applying vibrations may include applying vibrations to an inguinal canal of the human subject.

In other forms, applying vibrations may include applying vibrations to an ilioinguinal nerve of the human subject.

In yet another form, applying vibrations may include applying vibrations to an iliohypogastric nerve of the human subject.

In one example, applying vibrations may include applying vibrations to one or more of a genitofemoral nerve and a pudendal nerve of the human subject.

In some examples, coupling the vibration mechanism may include engaging the vibration mechanism with a scrotum of the human subject.

In another example, the method may include coupling a second vibration mechanism to another area of the pelvic region of the human subject.

In other examples, the method may include adjusting a frequency of the vibrations being applied, wherein the frequency is in a range of approximately 15 Hz to approximately 55 Hz.

In yet another example, applying the vibration device may include positioning a second vibration mechanism of the vibration device in a second target location of the pelvic region of the human subject.

In one aspect, applying the vibration device may include fastening a strap around the human subject.

In another aspect, the vibration mechanism may be supported by the strap.

In another aspect, applying the vibration device may include dressing the human subject with an undergarment.

In some aspects, the undergarment may be configured to support to the vibration mechanism.

In yet another aspect, the method may include inserting the vibration mechanism inside a pocket of the undergarment.

In some aspects, the method may include communicating with a controller using a remote communication device to adjust a frequency of a vibratory element of the vibration mechanism, wherein the controller is communicatively coupled to the vibratory element.

In other aspects, the vibration mechanism may include a second vibratory element.

In some forms, the controller may be configured to separately operate the vibratory element and the second vibratory element at different frequencies.

In one form, the garment may include a flexible band arranged for removably attaching to a pelvic region of the human subject.

In another form, the vibration mechanism may be coupled to the flexible band.

In other forms, a controller may be operatively coupled to the vibratory element.

In some forms, the controller may include a processor, a memory communicatively coupled to the processor and storing executable instructions that, when executed by the processor, causes the processor to receive data transmitted by a remote communication device, send a signal to the vibratory element, and adjust an intensity parameter of a vibration applied by the vibratory element.

### Definitions

As used herein, the terms “top,” “bottom,” “upper,” “lower,” “above,” and “below” are used to provide a relative relationship between structures. The use of these terms does not indicate or require that a particular structure must be located at a particular location in the apparatus.

Some examples may be described using the expression “coupled” and “connected” along with their derivatives. For example, some arrangements may be described using the term “coupled” to indicate that two or more elements are in direct physical or electrical contact. The term “coupled,” however, may also mean that two or more elements are not in direct contact with each other, but yet still co-operate or interact with each other. The examples described herein are not limited in this context.

As used herein, the term “pelvic region” or “pelvic/scrotal region” may be used to refer to right, left, or both sides of one or more of a lower pelvic region, scrotum, perineum, epididymis, vas deferens, testicle, testicular cord (or spermatic cord), cremasteric muscle(s), inguinal ring, inguinal canal, genitofemoral nerve, ilioinguinal nerve, iliohypogastric nerve, and pudendal nerve.

Examples of the present disclosure can include one or more of the following advantages.

The disclosed methods and devices may advantageously modulate the electrical or vibrational signals coming into the targeted areas (e.g., the testicular cord, including the testicular cremasteric muscles, inguinal canal, ilioinguinal nerve, the iliohypogastric nerve, and/or iliohypogastric nerve) and downregulate the sensitivity of the spinal cord/brain of the subject, such that the normal pain signals coming in will no longer be sufficient to register as pain.

In some examples, the disclosed methods and devices may be used to treat scrotal pain, chronic pelvic pain, meralgia paraesthetica, and cramping discomfort of irritable bowel syndrome.

In some examples, the disclosed methods and devices may provide alternatives to pharmacologic and surgical therapies.

In some examples, the disclosed devices may be integrated with a wearable garment or accessory to provide a discreet device that is low profile and quiet.

In some examples, the disclosed methods and devices may exhaust the ability of the targeted nerves to fire, which can occur with repeated stimulation.

In some examples, the disclosed methods and stimulation devices may be adapted to treat a patient population having subjects in a wide range of weight classes and with scrotal lengths and sizes.

In some examples, the wearable stimulation device of the present disclosure has an integrated, adjustable coupler that permits attaching to a scrotum according to the subject's scrotal physiology or comfort. The adjustable coupler can be tightened or loosened easily.

In another example, the stimulation device with a flexible electrode contact pad may be placed under a subject's panniculus or other areas of the body to effectively treat testicular pain. The electrodes are flexible and come in a variety of shapes and sizes to best suit a subject's physiology and comfort.

Other features and advantages of the present disclosure will be apparent from the following detailed description, figures, and claims.

### BRIEF DESCRIPTION OF THE DRAWINGS

FIG. 1 is a front view of an example stimulation device for treating testicular pain using electrical energy and vibration in accordance with the teachings of the present disclosure;

FIG. 2 is a front view of the stimulation device of FIG. 1 coupled to a pelvic/scrotal region of a human subject;

FIG. 3A is a front view of an example stimulation assembly for treating testicular pain in accordance with the teachings of the present disclosure;

FIG. 3B is a cross-sectional side view of a schematic drawing of a vibration mechanism of the assembly of FIG. 3A;

FIG. 4 is a back view of another example stimulation device for treating testicular pain in accordance with the teachings of the present disclosure;

FIG. 5 is a front view of the stimulation device of FIG. 4;

FIG. 6 is a front view of the stimulation device of FIG. 4 shaped to form a circular cuff;

FIG. 7 is a front view of the stimulation device of FIG. 4 coupled to a pelvic/scrotal region of a human subject;

FIG. 8 is a front view of a wearable system for treating testicular pain in accordance with the teachings of the present disclosure;

FIG. 9 is a front view of another wearable assembly for treating testicular pain in accordance with the teachings of the present disclosure;

FIG. 10 is a front view of another wearable assembly for treating testicular pain in accordance with the teachings of the present disclosure;

FIG. 11 is a front view of another wearable assembly for treating testicular pain in accordance with the teachings of the present disclosure;

FIG. 12 is a front view of another wearable assembly for treating testicular pain in accordance with the teachings of the present disclosure; and

FIG. 13 is a schematic drawing of a control system that can be used to perform operations in accordance with the teachings of the present disclosure.

### DETAILED DESCRIPTION

According to the present disclosure, devices and methods for non-invasively treating testicular pain may be adapted to a wide variety of physiologies of subjects to treat pain in the pelvic region, and specifically, near, above, and/or on the scrotum.

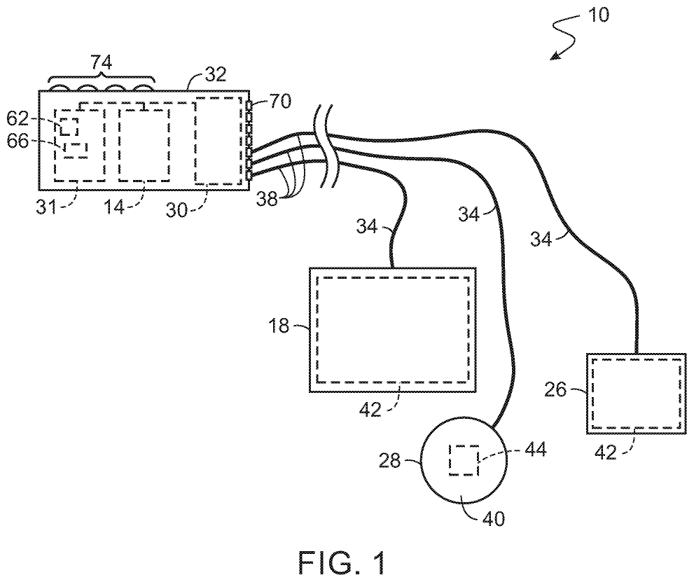

In FIG. 1, a first example stimulation device 10 is configured for external stimulation of a pelvic/scrotal region of a subject. The stimulation device 10 includes an electrical signal generator 14, a plurality of electrical contacts 18, 26, a battery 30 coupled to the generator 14, and a vibration mechanism 28. The generator 14, battery 30, and a controller 31 are disposed in a housing 32 of the device 10.

The first and second electrical contacts 18, 26 are electrically coupled to the generator 14 and the power source (e.g., a battery) 30, and the vibration mechanism 28 is coupled to the battery 30. In this example, the first and second electrical contacts 18, 26 are electrodes. The controller 31 is operatively coupled to the stimulation contacts 18, 26, 28 (e.g., both electrical and vibrational contacts) through a wired connection. Each of the electrical contacts 18, 26 includes a wire 34 having a plug 38 at a first end and a contact pad 42, which delivers the electrical energy to the subject, at an opposing end. Each wire 34 of the electrical contacts 18, 26 electrically couples the electrical contact 18, 26 to the generator 14. The vibration mechanism 28 also includes a wire and a plug 38 configured for connecting to the housing 32. In some examples, the vibration mechanism 28 may also be coupled to the generator 14.

The electrical contacts 18, 26 and vibration mechanism 28 are arranged for placement at various areas on or near a pelvic region 46 of a subject 48 to deliver stimulating energy, in the form, for example, of electrical pulses and vibrations, to the subject 48 to treat testicular pain. The electrical contacts 18, 26 and the vibration mechanism 28 may be used to apply electrical energy and vibration anywhere from an inguinal ring to a scrotum of a subject.

The vibration mechanism 28 includes a housing 40, a vibratory element 44 disposed in the housing 40, the wire 34, and the plug 38. The vibration mechanism 28 is configured to apply a massage-like input to a target nerve, muscle, or tissue. The vibratory element 44 is a DC motor that generates vibrations, such as an Eccentric Rotating Mass (“ERM”) vibration motor, which uses an unbalanced load to generate vibrations. However, in other examples, the vibratory element 44 may be a pancake or shaftless motor, a piezoelectric vibration generator, or other suitable means for producing vibrations. The vibratory element 44 is centrally located relative to the vibration mechanism housing 40. However, in this or other examples, the vibratory element 44 may be placed at any location within the housing 40.

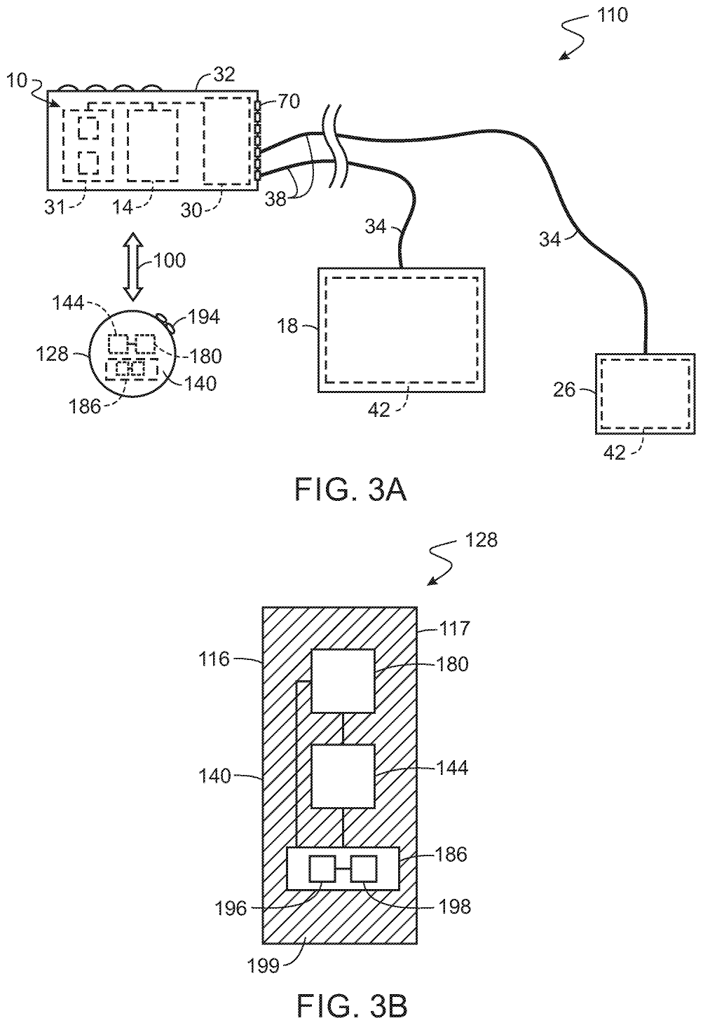

The vibration mechanism housing 40 is a thin, pliable membrane capable of protecting the internal components from sweat or other moisture, while also delivering vibrations to the subject 48. One or more layers of material (e.g., material 199 disposed in vibration mechanism housing 140 of FIG. 3B) may be disposed in the housing 40 that contribute (e.g., amplify, dampen, etc.) to a particular vibration massage or treatment. The housing 40 may be composed of and/or contain materials that are both capable of transmitting vibrations generated by the vibratory element 44 to the target treatment area of the subject 48 while also achieving a desirable feature, such as, for example, comfort, noise reduction, intensity, etc. For example, soft and pliable materials such as foam, cloth, or gel may be disposed between the vibratory element 44 and the subject 48 and that transmit vibrations while remaining comfortable to the subject 48. Increasing thickness of materials of the housing 40 or of materials disposed in the housing 40, for example, may reduce intensity and/or noise of the vibrations. Alternatively, reducing thickness of the materials of the housing 40 or of materials disposed in the housing 40, for example, may increase intensity and reduce the profile of the device 10. In some examples, an intensity of the vibrations may be modified by changing the shape of the housing 40 at the point of contact with the subject's body. In some examples, the housing 40 may be a silicone, plastic, or rubber material. In the illustrated example, the housing 40 is round with smooth surfaces. However, in other examples, the housing 40 may be another shape with textured or bumpy surfaces.

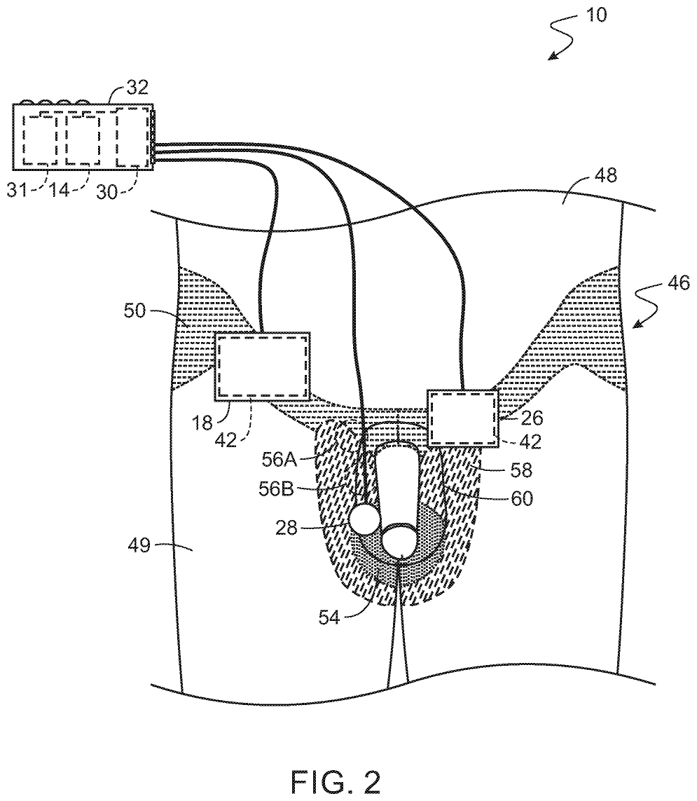

As shown in FIG. 2, the electrical contacts 18, 26 and vibration mechanism 28 are placed at various locations on a pelvic region 46 of a subject 48. The contact pad 42 of the first contact 18 is coupled to a first area 50 of the subject's pelvic region 46, and specifically adjacent to the inguinal canal of the subject's right side to stimulate an iliohypogastric nerve. The vibration mechanism 28 is coupled to a second area 54 of the subject's pelvic region 46, specifically on a right testicle of the scrotum 60, to stimulate a genitofemoral nerve of the subject 48. And the contact pad 42 of the second contact 26 is coupled to a third area 58 of the subject's pelvic region 46, and specifically adjacent to an inguinal canal of the subject's lower left side to stimulate both an ilioinguinal nerve and the iliohypogastric nerve.

The stimulation contacts 18, 26, 28 may be coupled to the subject at a combination of locations to best stimulate the testicular cord, including the testicular cremasteric muscles, inguinal canal, ilioinguinal nerve, the iliohypogastric nerve, and/or iliohypogastric nerve of the subject. In FIG. 2, first and second dashed areas 56A, 56B represent additional areas for placing one or more contacts 18, 26, 28 to treat testicular pain. It will be appreciated that the stimulation contacts 18, 26, 28 may be attached to any area of the pelvic region 46, such as, for example, along a circumference and length of the scrotum 60.

In the illustrated example of FIGS. 1 and 2, the first contact 18 has a large rectangular electrode pad 42 and the second contact 26 has a small rectangular electrode pad 42. The vibration mechanism 28 is puck shaped. The stimulation contacts 18, 26, 28 may be selected from a variety of contacts of various sizes and shapes depending on the treatment area and physiology of the subject. Once selected, the plug 38 of each contact 18, 26, 28 may then be coupled to a socket 70 of the housing 32 before initiating treatment. Each contact pad 42 of the electrical contacts 18, 26 has a contact surface that is configured to adhere to a skin surface of the subject 48. Similarly, a surface of the vibration mechanism 28 has a contact surface that is configured to adhere to the skin surface of the subject 48. The contact surfaces may include a textured surface, adhesive, or other material that is configured for self-adhering or otherwise attaching to a skin surface securely (e.g., by suction or friction). Additionally, the contact surface may be removed and reattached to a skin surface repeatedly.

Each of the wires 34 of the stimulation contacts 18, 26, 28 is removably coupled to a socket 70 formed in the housing 32. So configured, a subject can customize the stimulation device 10 by attaching a suitable contact of size and shape, and number of contacts. A plurality of sockets 70 are available for coupling additional contacts if more than three stimulation contacts are desired for treatment. In the illustrated example, the plug 38 of the vibration mechanism 28 electrically couples with any of the sockets 70 in the housing 32. However, in other examples, the vibration mechanism 28 may plug into a special socket 70 suitable only for vibration mechanisms.

While the stimulation device 10 of FIGS. 1 and 2 includes stimulation contacts 18, 26, 28 that each have a different shape and size, other configurations of the stimulation device 10 are possible. In some examples, the stimulation contacts may be the same size and shape, and may be a different shape than depicted in FIGS. 1 and 2. For example, to treat a wide area, one or more of the electrical contacts 18, 26 may be long rectangles that extend partially or entirely across a subject's pelvis. In other examples, the electrical contacts 18, 26 may be combined to create a variety of different geometries. In other examples, more than one vibration mechanism 28 may be coupled to the housing 32 to apply vibrations over a larger area or at different areas of the pelvic region 46.

Turning back to FIG. 1, the housing 32 of the device 10 houses the generator 14, power source 30, and control system 31, and defines a plurality of sockets 70 and a plurality of buttons 74. The control system 31 can communicate through signals (e.g., wired or wirelessly) to one or more components of the device 10. The controller 31 includes a processor 62 and a memory 66 that stores executable instructions that, when executed by the processor 62, can control certain settings (e.g., enable features, disable features, active mode, sleep mode, etc.) and parameters (e.g., frequency, intensity, time of delivery, delivery modes, etc.) of the stimulation device 10. For example, energy parameters may be adjusted to provide a deliverable electrical energy and vibrations having a desired intensity, frequency, amplitude, and duration. In some aspects, the control system 31 is a micro-processor based control system (or controller) that includes, e.g., hardware processor(s), memory module(s), and instructions executable as software code to cause the processor(s) to perform operations to control one or more components of the device 10. However, the control system 31 can also be realized as a mechanical, electro-mechanical, hydraulic, pneumatic, or other form of a control system or controller without departing from the scope of this disclosure.

The buttons 74 partially extend outside of the housing 32 for user interaction, and are configured to transmit a variety of different signals to the controller 31 to operate the stimulation device 10. For example, one or more buttons 74 may be pressed to turn on/off the device 10, increase or decrease a treatment parameter (e.g., pulse width, intensity, frequency, amplitude, time) of the electrical stimulation contacts 18, 26 and/or the vibration mechanism 28, or to adjust or program various settings and/or parameters of the stimulation device 10 (e.g., duration of delivering electrical energy, duration of delivering vibrations, setting alarms, providing modes, or programming treatment plans). In some examples, one or more dials, knobs, switches, may be used with or instead of the buttons 74 to operate the device 10.

The electrical contacts 18, 26 are configured to deliver electrical energy to the subject 48 via electrical pulses. The pulses can be adjusted by the subject or caregiver to a desired frequency, pulse width, amplitude, duration (i.e., time of the entire treatment), and mode (e.g., burst, continuous, other pattern) in accordance with a treatment plan and/or a comfort level of the subject 48.

For example, the frequency, or the number of electrical pulses delivered in one second, may be selected from a range of approximately 10 Hz or more (e.g., about 15 Hz or more, about 20 Hz or more, about 25 Hz or more, about 30 Hz or more, about 35 Hz or more, about 40 Hz or more, or about 45 Hz) to approximately 100 Hz or less (e.g., about 95 Hz or less, about 90 Hz or less, about 85 Hz or less, about 80 Hz or less, about 75 Hz or less, about 70 Hz or less, about 65 Hz or less, about 60 Hz or less, about 55 Hz or less, about 50 Hz or less, or about 45 Hz). A pulse width, or the elapsed time of a single pulse of energy, may be selected from a range of approximately 50 μs or more (e.g., about 60 μs or more, about 70 μs or more, about 80 μs or more, about 90 μs or more, about 100 μs or more, about 110 μs or more, about 120 μs or more, about 130 μs or more, about 140 μs or more, about 150 μs or more, about 160 μs or more, about 170 μs or more, about 180 μs or more, about 190 μs or more, or about 200 μs) to approximately 400 μs or less (e.g., about 390 μs or less, about 380 μs or less, about 370 μs or less, about 360 μs or less, about 350 μs or less, about 340 μs or less, about 330 μs or less, about 320 μs or less, about 310 μs or less, about 300 μs or less, about 290 μs or less, about 280 μs or less, about 270 μs or less, about 260 μs or less, about 250 μs or less, about 240 μs or less, about 230 μs or less, about 220 μs or less, about 210 μs or less, or about 200 μs). An amplitude of the pulse may be selected from a range of approximately 1 mA or more (e.g., about 5 mA or more, about 10 mA or more, about 15 mA or more, about 20 mA or more, about 25 mA or more, about 30 mA or more, about 35 mA or more, about 40 mA or more, about 45 mA or more, or about 50 mA) to approximately 100 mA or less (e.g. about 95 mA or less, about 90 mA or less, about 85 mA or less, about 80 mA or less, about 75 mA or less, about 70 mA or less, about 65 mA or less, about 60 mA or less, about 55 mA or less, or about 50 mA).

The vibration mechanism 28 can be adjusted by the subject, caregiver, or healthcare provider to a desired frequency or shift in frequency (e.g., gradual change or shift between two or more frequencies), duration (e.g., time of the entire treatment, time of applying vibrations, time of rest, and combination thereof), amplitude, and mode (e.g., burst, continuous, intermittent, or other pattern) in accordance with a treatment plan and/or a comfort level of the subject. The vibrational frequency may be selected from a range of approximately 10 Hz or more (e.g., about 15 Hz or more, about 20 Hz or more, about 25 Hz or more, about 30 Hz or more, about 35 Hz or more, about 40 Hz or more, or about 45 Hz) to approximately 100 Hz or less (e.g., about 95 Hz or less, about 90 Hz or less, about 85 Hz or less, about 80 Hz or less, about 75 Hz or less, about 70 Hz or less, about 65 Hz or less, about 60 Hz or less, about 55 Hz or less, about 50 Hz or less, or about 45 Hz).

In other examples, the housing 32 may include a user interface (e.g., a display screen, touchscreen, etc.) instead of or in addition to buttons 74 for operating and/or adjusting the parameters and settings of the stimulation device 10. As will be described in detail below, a subject 48 may adjust the parameters and settings of the stimulation device 10 through a personal electronic device, such as a smartphone, smartwatch, laptop, or other remote computer, that can communicate wirelessly (e.g., via Bluetooth) with the controller 31 disposed in the housing 32.

To treat testicular pain in the subject, a healthcare provider may first identify a location of testicular pain. After identifying the location of pain, the healthcare provider can recommend a target treatment area or location on the pelvic region 46 (including the scrotum 60) to treat the identified location of testicular pain. In some examples, the area or location of pain may be in a different area or location than the target treatment area or region.

The subject 48 or caregiver can apply the stimulation contacts 18, 26, 28 to various areas of the pelvic region 46 by adhering the contacts to the subject's skin. Once in place, the device 10 can be switched on, either wirelessly or locally via the buttons 74, and a treatment program may begin. Electrical energy and vibrations are delivered to the pelvic region 46 through the stimulation contacts 18, 26, 28 by operating the device 10. The subject 48 or caregiver can adjust the energy parameters of the deliverable electrical energy to operate and manage an electrical and vibrational stimulation treatment plan using the stimulation device 10. The stimulation device 10 can operate for a predetermined duration, e.g., 5 minutes to 60 minutes, or can operate in a single delivery of an electrical pulse, shock, or vibration to the target areas.

Externally coupling the electrical energy source using the device 10 of FIGS. 1 and 2, for example, includes attaching one or more electrical contacts 18, 26 to the subject 48. Coupling the vibration mechanism using the device 10 of FIGS. 1 and 2, for example, includes attaching or engaging the vibration mechanism 28 to the subject 48. Once attached, the energy source (e.g., the generator 14) delivers electrical energy and the vibratory element 44 delivers vibrations to one or more areas of the pelvic region 46. For example, using the device 10 of FIGS. 1 and 2, electrical energy is delivered to the subject 48 through multiple electrical pulses through the electrical contacts 18, 26, and vibrations are delivered to the subject 48 through the vibration mechanism 28.

The electrical energy source may be any suitable method that generates and/or transmits energy to a localized area. In one example, externally coupling an electrical energy source includes engaging an electrode with a skin surface of the subject 48. In other examples, this may include attaching a first electrode to a first area of the pelvic region 46 and attaching a second electrode to a second area of the pelvic region 46. In some examples, attaching an electrode may include adhering a first electrode to a region adjacent to an inguinal nerve or canal of the subject 48. In some examples, the electrode may be coupled to a scrotum 60 of the subject 48. In yet other examples, the energy source may be something other than an electrode, such as a defibrillator or similar energy delivery device.

Delivering energy to one or more areas of the pelvic region 46 may include delivering an electrical pulse to the inguinal canal, testicular cord, including the testicular cremasteric muscles, a genitofemoral nerve, an ilioinguinal nerve, and/or an iliohypogastric nerve of the subject 48. Delivering electrical energy may include delivering multiple electrical pulses over a period of time or delivering a single electrical shock to one or more areas of the pelvic region 46. In some examples, delivering multiple electrical pulses may include constant stimulation, infrequent bursts, or a combination of modes and methods.

An example electrical treatment plan for one or more electrical contacts may include delivering a first electrical pulse at a lower frequency for a period of time, and gradually or periodically, increasing the frequency. Another example treatment may include applying intermittent pulses so that the electrical contact delivers pulses at a first frequency for a first time period, and stops for a second time period, and delivers an electrical pulse at the first or different frequency for a third time period after rest. Another example treatment may be fully customizable by the subject. These treatment plans may be done in unison or independently from the other electrical contacts and/or vibration mechanisms.

An example vibrational treatment plan may include applying a vibration at a lower frequency for a period of time, and gradually or periodically, increasing the frequency. Another example treatment may include applying intermittent vibrations so that the vibration mechanism applies vibrations at a first frequency for a first time period, and stops for a second time period, and applies vibrations at the first or different frequency for a third time period after rest. Another example treatment may be fully customizable by the subject. The stimulation device 10 can operate for a predetermined duration at one or more frequencies, e.g., 5 minutes at 25 Hz, 5 minutes at 50 Hz, etc. In some examples, the device 10 may include a second vibration mechanism so that a first vibration mechanism can be placed on a right side of the pelvic region 46, and close to the iliohypogastric nerve 50, and a second vibration mechanism is placed below a scrotum 60 and close to the genitofemoral nerve 54. A wide variety of combinations of the device 10 are possible.

These treatment plans may be performed in unison or independently from the other electrical contacts and vibration mechanisms. In some examples, the controller of the stimulation devices may be configured to selectively adjust the device parameters for each stimulation contact. For example, if a first area of the pelvic region requires more intense treatment than a second treated area, the user can adjust the parameters and/or settings of the electrical contacts and/or vibration mechanisms so that the first electrical contact and vibration mechanism deliver an intense stimulation treatment to a first region and the second electrical contact delivers a less intense stimulation treatment.

In the example shown in FIGS. 1 and 2, the vibration mechanism 28 is powered by the battery 30 in the device housing 32. However, in other examples, such as the example shown in FIGS. 3A and 3B, a vibration mechanism 128 includes a battery 180 disposed within a housing 140 of the vibration mechanism 128. In some examples, the vibration mechanism 128 can communicate wirelessly with the controller 31 of the device 10 through wireless communication signals 100. In other examples, the vibration mechanism 128 may include a separate controller 186 that can be operated independently from the controller 31 of the device 10.

Referring to FIGS. 3A and 3B, a second example stimulation assembly 110 includes the stimulation device 10 of FIGS. 1-2 and a wireless vibration mechanism 128. The stimulation device 10 is similar to the stimulation device 10 of FIGS. 1 and 2, and the electrical energy source includes two electrical contacts 18, 26 for delivering electrical energy to a subject 48 and a vibration mechanism 128 for delivering vibrations to the subject 48. Thus, for ease of reference, and to the extent possible, the same or similar components of the assembly 110 will retain the same reference numbers as outlined above with respect to the device 10, although the reference numbers will be increased by 100.

The vibration mechanism 128 includes the housing 140, a vibratory element 144, the battery 180, and a communication module (e.g., via transmitters and receivers) or controller 186. The vibratory element 144, battery 180, and controller 186 are disposed in the housing 140 and are electrically connected so that the controller 186 can operate the vibratory element 144 and the battery 180 can provide power to the vibratory element 144 and the controller 186. To couple the vibration mechanism 128 to the subject 48, one or more surfaces of the housing 140 may adhere to the skin of the subject. In other examples, the user may hold the vibration mechanism 128 against the treatment area. In some examples, a retractable cord for clipping onto clothing of the subject may be at least partially stored in the housing 140.

In some examples, the vibration mechanism 128 and the controller 31 of the stimulation device 10 are in wireless communication. In this example, the vibration mechanism 128 is configured to send signals to, and receive signals from, the controller 31 of the device 10, and the controller 31 operates the vibration mechanism 128.

In some examples, the controller 186 of the vibration mechanism 128 may be independently controlled by one or more buttons 194 or through wireless communication with a personal electronic device, such as a smartphone, smartwatch, laptop, or other remote computer. The control system 186 can communicate through signals (e.g., wired or wirelessly) to one or more components of vibration mechanism 128. The control system 186 may also communicate to the controller 31 of the device 10. The controller 186 includes a processor 196 and a memory 198 that stores executable instructions that, when executed by the processor 196, can control certain settings and parameters of the vibration mechanism 128. For example, treatment parameters may be adjusted to provide vibrations having a desired intensity, frequency, and duration. In some aspects, control system 186 is a micro-processor based control system (or controller) that includes, e.g., hardware processor(s), memory module(s), and instructions executable as software code to cause the processor(s) to perform operations to control one or more components of the vibration mechanism 128. A plurality of buttons 194 are configured to receive input to operate and control the vibration mechanism 128. In some examples, the controller 186 may be operated (e.g., switch the device on or off, adjust vibration intensity, adjust vibration timing, etc.) by pressing one or more of the buttons 194. However, the control system 186 can also be realized as a mechanical, electro-mechanical, hydraulic, pneumatic, or other form of a control system or controller without departing from the scope of this disclosure. One or more vibrational treatment programs may be stored in the memory of the processor can be accessed and adjusted.

The buttons 194 partially extend outside of the housing 140 for user interaction, and are configured to transmit a variety of different signals to the controller 186 to operate the vibration mechanism 128. For example, one or more buttons 194 may be pressed to turn on/off the vibration mechanism 128, increase or decrease a treatment parameter (e.g., intensity, frequency, time, etc.), or to adjust or program various settings of the vibration mechanism 128 (e.g., duration of delivering vibrations, setting alarms, providing modes, or programming treatment plans). In some examples, one or more dials, knobs, switches, may be used with or instead of the buttons 194 to operate the vibration mechanism 128.

In some examples, the housing 140 of the vibration mechanism 128 may include a user interface (e.g., a display screen, touchscreen, etc.) instead of or in addition to buttons 194 for operating and/or adjusting the treatment parameters and settings of the vibration mechanism 128. For example, a subject, caregiver, or healthcare provider may adjust the treatment parameters and settings of the vibration mechanism 128 through a personal electronic device, such as a smartphone, smartwatch, laptop, or other remote computer that can communicate wirelessly (e.g., via Bluetooth) with the controller 186 disposed in the housing 140. In some examples, the healthcare provider may monitor the subject's usage of the device and adjust the treatment parameters from a remote location.

While the vibration mechanism 128 shown in FIG. 3B has been illustrated as having the vibratory element 144 centrally disposed in the housing 140 between parallel sides 116, 117, in other examples, the vibratory element may be disposed closer to one side of the housing 140. In this example, engaging this one side of the housing with the subject may apply stronger vibrations than engaging the other side of the housing with the subject. In other examples, the vibration mechanism 128 may include a first vibratory element adjacent to one side of the housing and a second vibratory element adjacent to the other side of the housing. In this case, either side or surface of the housing can be coupled to (or engaged with) the subject to apply vibrations interchangeably.

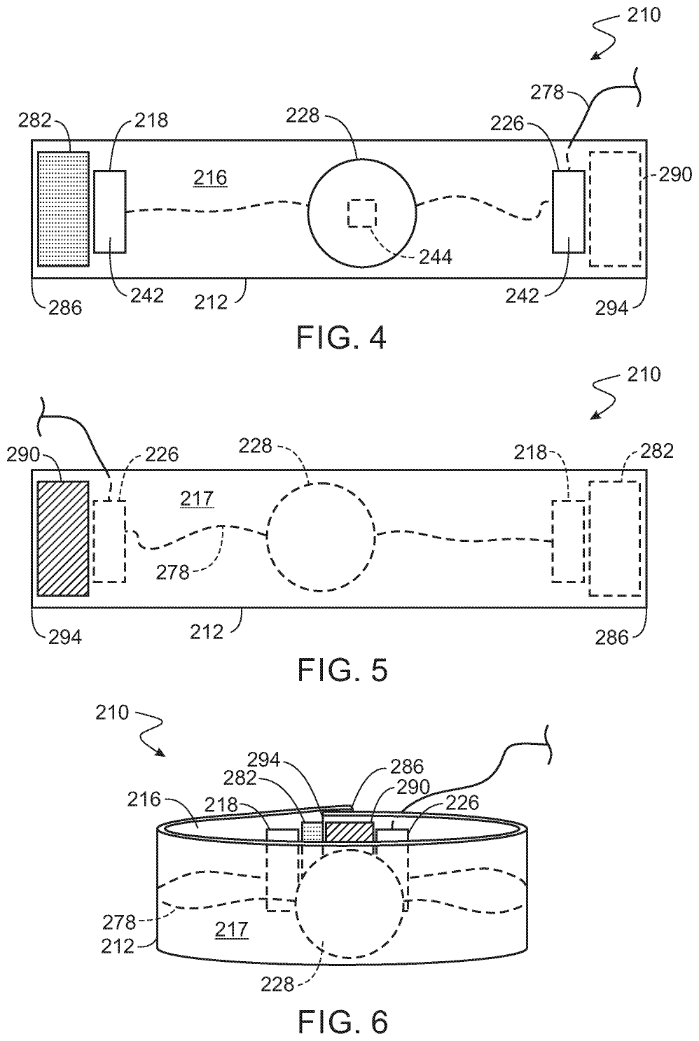

Referring now to FIGS. 4-7, an electrical stimulation device 210 includes an electrical energy source and vibration source for delivering electrical and vibrational energy to a pelvic region 46 of a subject 48. The stimulation device 210 of FIGS. 4-7 is similar to the stimulation device 10 of FIGS. 1 and 2, and includes two electrical contacts 218, 226 for delivering electrical energy and a vibration mechanism 228 for delivering vibrations to the subject 48. Thus, for ease of reference, and to the extent possible, the same or similar components of the device 210 of FIGS. 4-7 will retain the same reference numbers as outlined above with respect to the device 10 of FIGS. 1 and 2, although the reference numbers will be increased by 200.

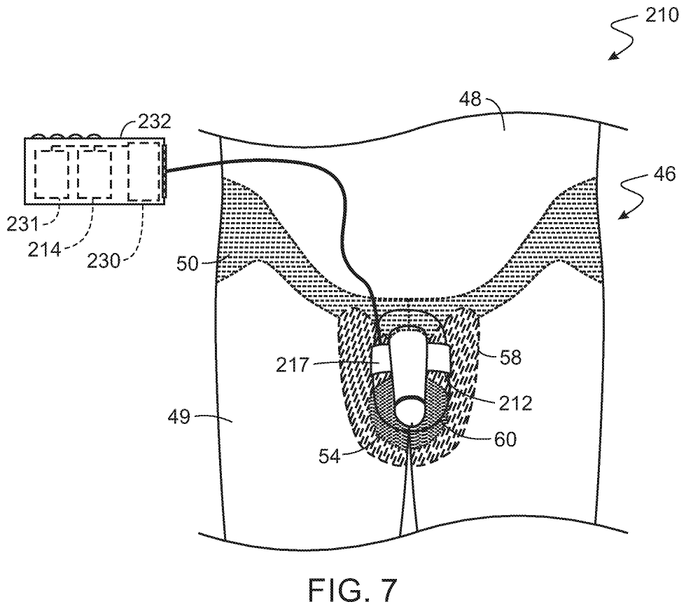

The stimulation device 210 includes a flexible body 212 including an interior surface 216 (FIG. 4), an exterior surface 217 (FIG. 5), two electrodes 218, 226, and a vibration mechanism 228 attached to (e.g., embedded between) one or more layers of the body 212. A housing 232 contains an electrical signal generator 214, a battery 230, and a control system 231 (FIG. 7). In FIG. 4, each contact pad 242 of the electrodes 218, 226 is exposed on the interior surface 216 of the body 212, and in FIG. 5, the electrodes 218, 226 are covered by a layer of material on the exterior surface 217. The vibration mechanism 228 has an exterior surface that can engage the subject directly 48 or may be covered by one or more layers of material. As shown in FIG. 6, the body 212 can be arranged into a cuff that is sized to fit at least partially around a scrotum 60 of the subject 48. The cuff is arranged so that the interior surface 216 engages the scrotum 60 of the subject 48, as shown in FIG. 7. In this way, the cuff can stimulate the testicular cord and cremasteric muscle of the subject 48 to treat testicular pain.

The electrodes 218, 226 and vibration mechanism 228 are spaced relative to each other in a way that when the cuff is positioned on the scrotum 60, the contact pads 242 of the electrodes 218, 226 and vibration mechanism 228 engage different areas of the subject's scrotum 60. When arranged on the scrotum 60, the first and second electrodes 218, 226 are placed on a back side of the scrotum 60, and the vibration mechanism 228 is placed on a front side of the scrotum 60. However, the cuff may be rotated to stimulate different areas of the scrotum 60. In other examples, the body 212 may include additional electrodes or vibration mechanisms placed variously along a length of the body 212. The electrodes 218, 226 and vibration mechanism 228 are electrically coupled by a wire 278 embedded between one or more layers of the body 212. The wire 278 extends externally from the body 212 to connect with a housing 232 (FIG. 7). The device 210 may be operated in the same or similar manner as the device 10 by adjusting the settings of the device 210 and/or treatment parameters using the buttons 274 and/or the control system 231. In some examples, the vibration mechanism 228 may be battery powered.

The device 210 includes an adjustable coupler to treat a range of scrotal sizes. The coupler includes a first coupling 282 disposed at a first end 286 of the body 212 and a second coupling 290 disposed at a second end 294 of the body 212. The first and second couplings 282, 290 are disposed on opposite surfaces 216, 217 of the body 212 such that when the first and second ends 286, 294 of the body 212 are joined to form the cuff, the first and second couplings 282, 290 overlap and mate. In the illustrated example, the coupler is a hook-and-loop fastener where the first coupling 282 includes a plurality of hooks that are arranged to fasten to a plurality of loops of the second coupling 290.

In some examples, the body 212 of the stimulation device 210 may have a microcontroller, power source, and/or generator integrated into one or more layers of the body 212. In such an example, the body 212 may be rechargeable and may be configured to directly communicate with a user's personal electronic device for operation.

In some examples, the device 210 may include more or fewer electrodes and more vibration mechanisms that can be activated or deactivated depending on the treatment plan to treat testicular pain.

In some examples, the body 212 of the stimulation device 210 may be arranged differently so that the cuff is formed using, for example, a tie, buckle, button, snap, or other suitable male-female mating structure or fastener. In other examples, the body 212 is circular and only needs slight adjustment after placing the device 210 around the scrotum 60 of the subject. In this case, rather than coupling opposing ends of a rectangular strip embedded with electrodes and a vibration mechanism, one example device may be preconfigured as a cuff to receive the scrotum into a central opening of the cuff. In some examples, the body 212 may be made of a memory-shape material that can hold a new shape in response to being manipulated and shaped (e.g., bent, twisted, etc.).

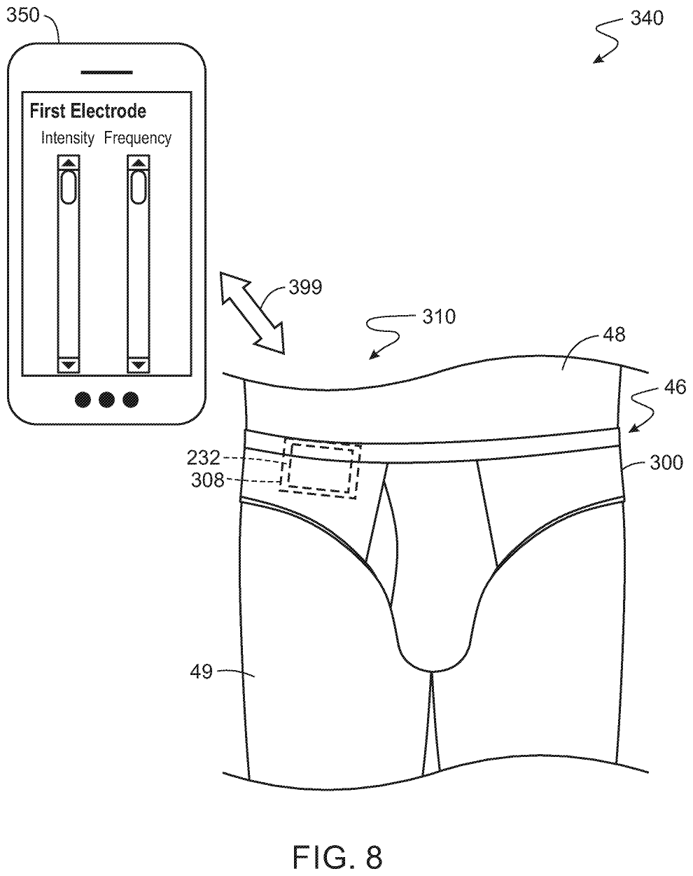

In some examples, the stimulation device 10, 210 may be incorporated into a garment, such as underwear, as illustrated in FIG. 8. For example, an underwear pouch that covers a subject's penis and scrotum may include a plurality of spaced electrodes and vibration mechanisms that can be selectively operated to deliver electrical and vibrational stimulation to various locations of the pelvic region. Other regions of the underwear may also incorporate electrical energy and vibrational sources.

Referring now to FIG. 8, a treatment system 340 includes a wearable assembly 310, a smartphone 350, and a stimulation device (hidden from view), such as the devices 10, 210 or assembly 110 described herein. The stimulation device is removably coupled to a garment 300, which in this example is underwear. The undergarment 300 has a built-in pocket 308 sized to receive the housing (e.g., housing 232 of a device 210) and accommodates a cuff, contacts, and wires. The garment 300 fits over the stimulation device 10, 210 attached to the subject 48 so that the subject 48 may wear and easily operate the stimulation device 10, 210 throughout the course of a day.

The devices 10, 210 described herein may be operated using a personal electronic device 350, such as a smartphone, smartwatch, laptop, or other remote computer, as shown in the example treatment system 340 of FIG. 8. The treatment system 340 includes an application stored on a personal electronic device 350. Using wireless communication, such as via Wi-Fi, the subject 48 can control the stimulation device 210 remotely via wireless communication signals 399 rather than retrieving the housing 232 from the pocket 308 of the garment 300 and operating the device manually (although, in some examples, manual operation is optional). In this example, the subject 48 can use an application stored on the personal electronic device 350 to operate and adjust various features of the stimulation device. For example, the subject 46 can adjust the settings and parameters of the stimulation device to deliver a particular stimulating treatment to the pelvic region 46. The application may also be programmable so that the subject 48 may create and store a treatment plan for repeated use. The application may also be remotely accessed by a health care provider so that the health care provider may adjust the settings and/or parameters of the device 10, 210 and create and/or modify treatment programs customized for the subject 48.

In the illustrated example of FIG. 8, the personal electronic device 350 displays two energy parameters for controlling an energy output of a first electrode. However, in other examples, each electrode and vibration mechanism may have more or fewer adjustable parameters and settings. Additionally, in some examples, the energy parameters of the electrodes and/or vibration mechanisms may be adjusted collectively.

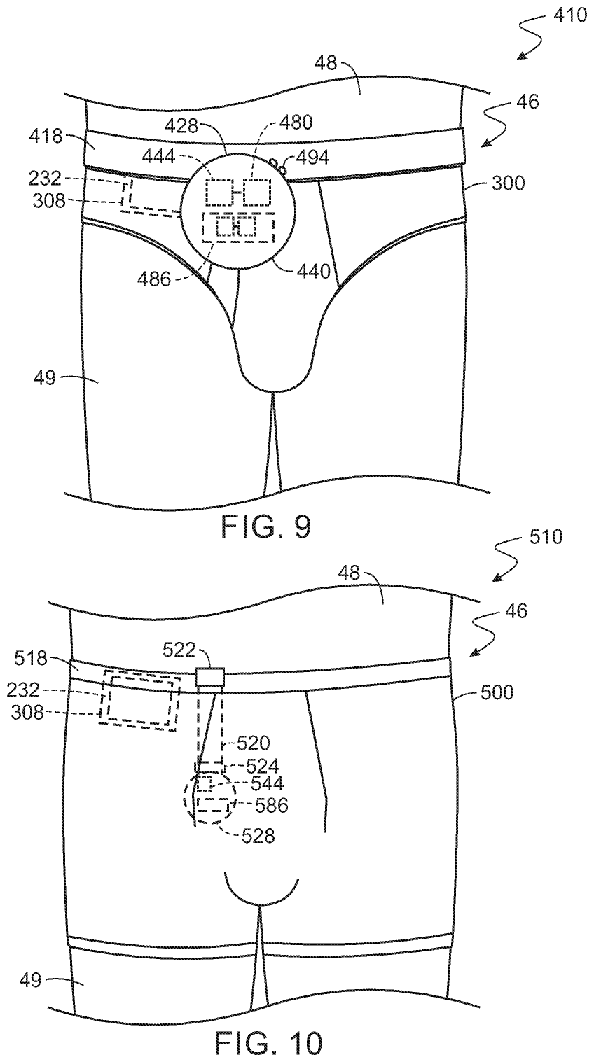

While the stimulation devices and assemblies 10, 110, 210 of FIGS. 1-7 have been described as having a vibration mechanism 28, 128, 228 that adheres or engages directly to a subject's skin, in some examples, a vibration mechanism may hang from a belt or strap, as shown in FIGS. 9-11.

Referring to FIG. 9, a stimulation assembly 410 includes a vibration mechanism 428 and the device 10, 210 for delivering vibrational and electrical energy to a pelvic region 46 of a subject 48 to treat testicular pain. The stimulation assembly 410 is similar to the stimulation assembly 110 of FIGS. 3A and 3B, and includes one or more electrical contacts for delivering electrical energy and the vibration mechanism 428 for delivering vibrations to the subject 48. Thus, for ease of reference, and to the extent possible, the same or similar components of the assembly 410 of FIG. 9 will retain the same reference numbers as outlined above with respect to the assembly 110 of FIGS. 3A and 3B, although the reference numbers will be increased by 300.

In FIG. 9, the vibration mechanism 428 is coupled to the subject 48 by an adjustable strap or belt 418 extending around a waist or hips of the subject 48. The vibration mechanism 428 is positioned close to a iliohypogastric nerve on a right side of the subject's pelvic region 46. The user may lower the belt 418 (or strap the belt 418 around the subject's thigh 49) to position the vibration mechanism 428 close to an ilioinguinal nerve or close to a genitofemoral nerve.

The vibration mechanism 428 is puck-shaped and can be positioned at any location on the belt 418 by sliding the vibration mechanism 428 relative to the belt 418 and/or by clipping the vibration mechanism 428 to the belt 418 at a desired location. The vibration mechanism 428 is fastened (e.g., clipped, snapped, buckled, hook-and-loop mated, etc.) to the belt 418 so that the vibration mechanism 428 is positioned in a desired target area of treatment. In some examples, the user may rotate the belt 418 relative to their body and vibration mechanism 428 to position the vibration mechanism 428 at a target area. In some examples, the vibration mechanism 428 is rotatable relative to the belt 418 to position the vibration mechanism 428. In some examples, the vibration mechanism 428 may be adjusted vertically (relative to the orientation of FIG. 9) so that the belt 418 remains around the user's waist or hips while the vibration mechanism 428 is positioned at a lower portion of the subject's pelvic region 46. While not illustrated in FIG. 9, the vertical positioning of the vibration mechanism 428 may be adjusted using a retractable cord or additional strap that is coupled to the vibration mechanism 428. In another example, the belt 418 may be fastened to an upper thigh 49 of the subject 48. The belt 418 is adjustable to accommodate different body sizes and different body parts. The belt 418 may be a pliable or stretchy material.

In FIG. 10, another example stimulation assembly 510 includes a vibration mechanism 528 and the device 10, 210 for applying vibrational and electrical energy to a pelvic region 46 of a subject 48 to treat testicular pain. The stimulation assembly 510 is similar to the stimulation assembly 110 of FIGS. 3A and 3B, and includes one or more electrical contacts for delivering electrical energy and a vibration mechanism 528 for delivering vibrations to the subject 48. Thus, for ease of reference, and to the extent possible, the same or similar components of the assembly 510 of FIG. 10 will retain the same reference numbers as outlined above with respect to the assembly 110 of FIGS. 3A and 3B, although the reference numbers will be increased by 400.

The assembly 510 is configured to be worn under a garment 500, such as shorts or briefs, and the vibrational mechanism 528 is attached to a waistband of the garment 500 or a belt 518. The vibration mechanism 528 of the assembly 510 is coupled to a strap 520, and is interiorly placed relative to the garment 500 worn by the subject 48. The strap 520 has an adjustment mechanism 524 that allows the strap 520 to extend or contract based to a desired length. At a distal end 522, the strap 520 forms a hook so that an underside surface of the hook engages an exterior surface of the belt 518 or waistband of the garment 500 and secures the vibration mechanism 528 to the subject's clothing. The underside of the hook 522 has a grippable surface that securely attaches the strap 520 to the garment 500 and/or belt 518, but is configured for easily disengaging from the garment 500 and/or belt 518 to reposition or remove the vibration mechanism 528. The vibration mechanism 528 may be placed directly on the skin of the subject 48, or may be placed between a layer of material and the garment 500.

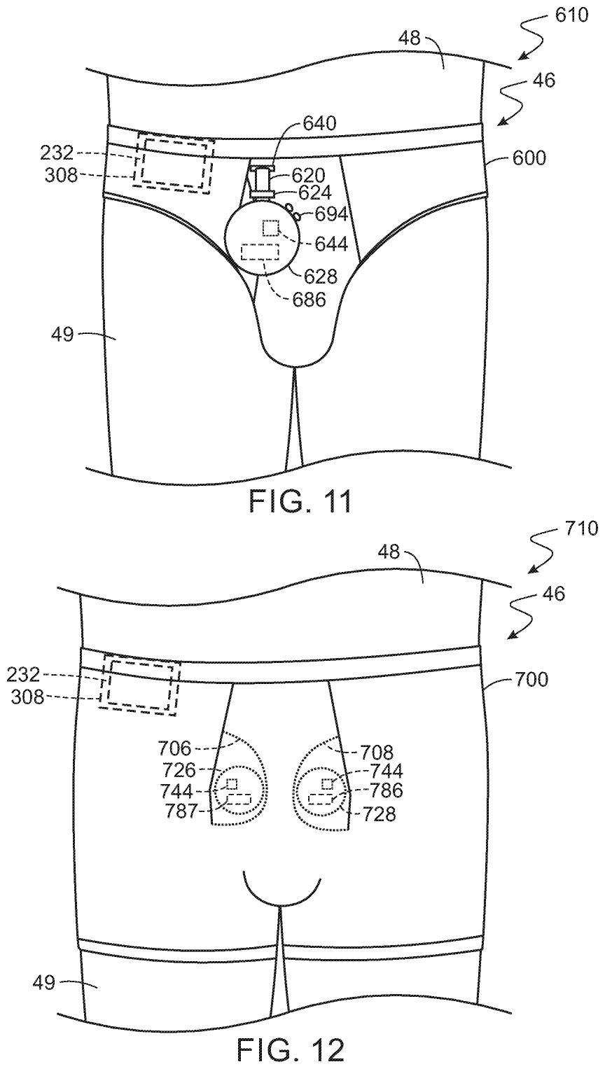

Referring to FIG. 11, another example stimulation assembly 610 includes a vibration mechanism 628 and the device 10, 210 for applying vibrational and electrical energy to a pelvic region 46 of a subject 48 to treat testicular pain. The stimulation assembly 610 is similar to the stimulation assembly 110 of FIGS. 3A and 3B, and includes one or more electrical contacts for delivering electrical energy and a vibration mechanism 628 for delivering vibrations to the subject 48. Thus, for ease of reference, and to the extent possible, the same or similar components of the assembly 610 of FIG. 11 will retain the same reference numbers as outlined above with respect to the assembly 110 of FIGS. 3A and 3B, although the reference numbers will be increased by 500.

The stimulation assembly 610 is coupled to a different garment 600, such as underwear, and is configured to apply vibrations over a layer of material (e.g., the underwear material). The garment 600 includes a reinforced opening 640 that is sized to receive a distal end of a strap 620 to hook onto the undergarment 600. In this example, the garment 600 is configured for receiving the vibrational mechanism 628, and treating testicular pain, on a right side of the subject 48. In other examples, the garment 600 may be configured for treating testicular pain on a left side of the subject 48 by providing a reinforced opening on the left side of the garment 600. In some examples, the garment 600 may include two or more openings for repositioning the vibration mechanism 628 as needed.

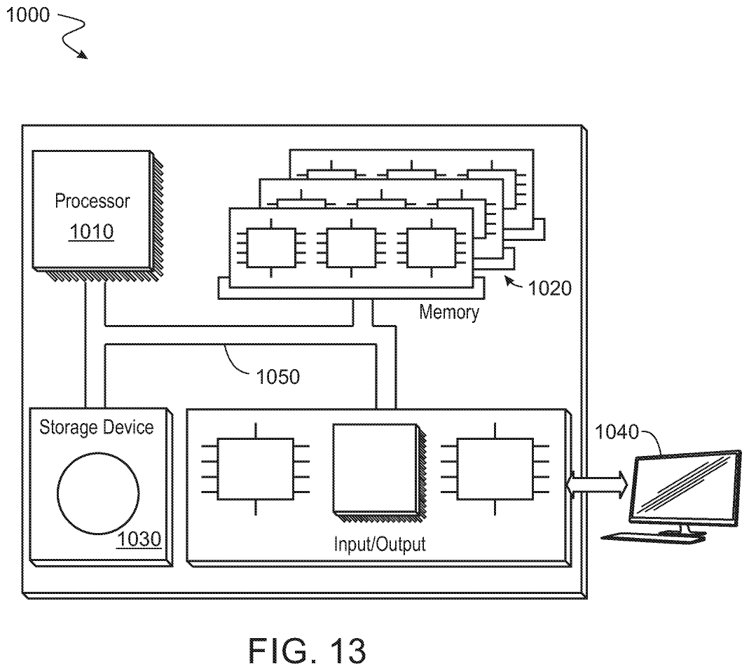

While the stimulation devices and assemblies 10, 110, 310, 410, 510, 610 of FIGS. 1-11 have been depicted as having a single vibration mechanism 28, 128, 228, 428, 528, 628, in some examples, a stimulation device or assembly may include more than one vibration mechanism, as shown in FIG. 12.

In FIG. 12, an example stimulation assembly 710 includes two vibration mechanisms 726, 728 and the device 10, 210 for applying vibrational and electrical energy to a pelvic region 48 of a subject 46 to treat testicular pain. The stimulation assembly 710 is similar to the stimulation assembly 110 of FIGS. 3A and 3B, and includes one or more electrical contacts for delivering electrical energy and a vibration mechanism for delivering vibrations to the subject 48. Thus, for ease of reference, and to the extent possible, the same or similar components of the assembly 710 of FIG. 12 will retain the same reference numbers as outlined above with respect to the assembly 110 of FIGS. 3A and 3B, although the reference numbers will be increased by 600.

In FIG. 12, the wearable assembly 710 includes a garment 700, which in this example is underwear, and two vibration mechanisms 726, 728 coupled to the garment. The undergarment 700 has two, internally built-in pockets 706, 708, with each pocket 706, 708 being sized to receive one of the vibration mechanisms 726, 728. Each of the vibration mechanisms 726, 728 includes a vibratory element 744, a battery, and a controller or communication module 786, 787. Accordingly, the vibration mechanisms 726, 728 can be programmed to apply different vibrational massage treatments to the different areas of the pelvis 46. The vibration mechanisms 726, 728 may also be programmed to apply the same vibrational massage treatment.

Each vibration mechanism 726, 728 of the stimulation assembly 710 may be activated or deactivated depending on the treatment plan to treat testicular pain. In some examples, each of the controllers 786, 787 of the vibration mechanisms 728, 726 may be configured to selectively adjust the treatment parameters for the respective vibratory element 744. For example, if a first area of the pelvic region 46 requires more intense treatment than a second treated area, the user can adjust the settings and/or parameters of the vibration mechanism 726 so that the first vibration mechanism 726 delivers an intense massage vibration treatment to the first region, and adjust the settings and/or parameters of the vibration mechanism 728 so that the second vibration mechanism 728 delivers a less intense massage vibration treatment.

The subject 48 may wear and easily operate the stimulation device 310 throughout the course of a day. For example, the user can retrieve one or more of the vibration mechanisms 726, 728, operate it (e.g., turn on/off, adjust vibration parameters via the buttons), and insert the one or more vibration mechanism 726, 728 back into the pockets 706, 708. In other examples, the undergarment 700 may include more or fewer pockets. For example, the underwear pouch that covers a subject's penis and scrotum may include a plurality of pockets for receiving a plurality of vibration mechanisms that can be selectively operated to deliver vibrations to various locations of the pelvic region 46. In another example, the pockets 706, 708 may be large enough to receive two or more vibration mechanisms.

While the stimulation device 710 of FIG. 12 has been described as having two vibration mechanisms 726, 728, in other examples, a wearable stimulation device or assembly may include one or more than two vibration mechanisms that fit into a corresponding number of pockets disposed in the same or different areas relative to the pelvic region of the garment. In other examples, the vibration mechanisms may be sewn into the undergarment for permanent fixation.

While the stimulation device 710 of FIG. 12 has been described as having two separate and independently controlled vibration mechanisms 726, 728, in other examples, a stimulation assembly or device may include two or more vibration mechanisms that are connected and do not operate independently. For example, one or more vibration mechanisms may be electrically coupled to the device 10, 210. In another example, the vibration mechanisms may be electrically connected by a wire that can stretch or extend between the pockets of the garment.

While the vibration mechanisms 28, 128, 228, 428, 528, 628, 726, 728 of the stimulation devices and assemblies 10, 110, 210, 310, 410, 510, 610, 710 of FIGS. 1-12 have been depicted or described as smooth-surfaced pucks, in other examples, a vibration mechanism may have a different shape and the housing may have different surface treatment.

While the vibration mechanisms 28, 128, 228, 428, 528, 628, 726, 728 of the stimulation devices and assemblies 10, 110, 210, 310, 410, 510, 610, 710 of FIGS. 1-12 have been described as having one vibratory element, in other examples, a vibration mechanism of a stimulation device may have two or more vibratory elements disposed in a vibration mechanism housing or in the device housing.

In some examples, multiple vibratory elements share electrical power with one another, and in other examples, the vibratory elements may connect to separate batteries. The controller of the device or of the vibration mechanism may be coupled to each vibratory element separately, or in other examples, the controller may be coupled to only one vibratory element, which in turn is coupled to the other vibratory elements, so that the controller operates the vibratory elements in unison.

While the stimulation devices and assemblies 310, 410, 510, 610, 710 of FIGS. 8-12 are coupled to, or embedded in, garments (e.g., pants, underwear, briefs, belts), in other examples, a stimulation device may be incorporated in different wearable items. In other examples, the stimulation device may be a handheld device.

In some examples, the stimulation devices and assemblies 10, 110, 210, 310, 410, 510, 610, 710 may incorporate heating or cooling elements that can be operated to deliver heat or a cooling sensation in addition to or instead of applying vibrations or delivering electrical energy. In some examples, the vibratory element of the vibration mechanism is configured to emanate heat or a cooling sensation while applying vibrations.

In some examples, treating testicular pain includes delivering electrical energy to a subject 48 and the devices and assemblies 10, 110, 210, 310, 410, 510, 610, 710 may include one or more of the following methodologies for delivery electrical energy: transcutaneous electrical nerve stimulation, Russian stimulation, neuromuscular electrical stimulation (NMES), inferential current (IFC) electrical stimulation, high voltage electrical stimulation, iontophoresis, or a combination thereof.

In some examples, the stimulation devices and assemblies 10, 110, 210, 310, 410, 510, 610, 710 may be configured for delivering electrical, radiation, magnetic, conducted energy, or a combination thereof to effectively treat testicular pain.

With reference to FIGS. 1-12, an example operation of the stimulation devices and assemblies 10, 110, 210, 310, 410, 510, 610, 710 can be performed to treat testicular pain. One or more steps of the example operation can be performed by or with the control system 31, 186, 231, 486, 586, 686, 786, 787 (e.g., with input from a human operator). For instance, the operation can include adjusting a pulse intensity, setting a timer for treatment, sending results to a healthcare provider, etc.

FIG. 13 shows a schematic drawing of a control system 1000 that can be used, e.g., as control system 31, 186, 231, 486, 586, 686, 786, 787, to perform operations according to the present disclosure. Some or all of the example control system 1000 can be implemented as cloud-based system and/or service, alone or in combination with other portions of the example control system 1000 that can be implemented to execute. The controller 1000 is intended to include various forms of digital computers, such as printed circuit boards (PCB), processors, digital circuitry, or otherwise. Additionally, the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

The controller 1000 includes a processor 1010, a memory 1020, a storage device 1030, and an input/output device 1040. Each of the components 1010, 1020, 1030, and 1040 are interconnected using a system bus 1050. The processor 1010 is capable of processing instructions for execution within the controller 1000. The processor may be designed using any of a number of architectures. For example, the processor 1010 may be a CISC (Complex Instruction Set Computers) processor, a RISC (Reduced Instruction Set Computer) processor, or a MISC (Minimal Instruction Set Computer) processor.

In one implementation, the processor 1010 is a single-threaded processor. In another implementation, the processor 1010 is a multi-threaded processor. The processor 1010 is capable of processing instructions stored in the memory 1020 or on the storage device 1030 to display graphical information for a user interface on the input/output device 1040.

The memory 1020 stores information within the control system 1000. In one implementation, the memory 1020 is a computer-readable medium. In one implementation, the memory 1020 is a volatile memory unit. In another implementation, the memory 1020 is a non-volatile memory unit.

The storage device 1030 is capable of providing mass storage for the controller 1000. In one implementation, the storage device 1030 is a computer-readable medium. In various different implementations, the storage device 1030 may be a floppy disk device, a hard disk device, an optical disk device, a tape device, flash memory, a solid state device (SSD), or a combination thereof.

The input/output device 1040 provides input/output operations for the controller 1000. In one implementation, the input/output device 1040 includes a keyboard and/or pointing device. In another implementation, the input/output device 1040 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, for example, in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random-access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, solid state drives (SSDs), and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) or LED (light-emitting diode) monitor for displaying information to the user and a keyboard and a pointing device such as a mouse or a trackball by which the user can provide input to the computer. Additionally, such activities can be implemented via touchscreen flat panel displays and other appropriate mechanisms.

The features can be implemented in a control system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

While this specification contains many specific implementation details, these should not be construed as limitations on the scope of any disclosure or of what may be claimed, but rather as descriptions of features that may be specific to particular examples of particular disclosures. Certain features that are described in this specification in the context of separate examples can also be implemented in combination in a single example. Conversely, various features that are described in the context of a single example can also be implemented in multiple examples separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the examples described herein should not be understood as requiring such separation in all examples, and it should be understood that the described program components and systems can generally be integrated together in a single product or packaged into multiple products.

Particular examples of the subject matter have been described. Other examples are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method of treating testicular pain of a subject, the method comprising:
coupling an electrical energy source to a pelvic region of the subject;
coupling a vibration mechanism to the pelvic region of the subject;
delivering electrical energy to the pelvic region; and
applying vibrations to the pelvic region.

2. The method of claim 1, wherein coupling the electrical energy source comprises externally engaging the electrical energy source with a scrotum of the subject.

3. The method of claim 1, wherein coupling the vibration mechanism comprises engaging the vibration mechanism with a scrotum of the subject.

4. The method of claim 1, wherein coupling the electrical energy source and coupling the vibration mechanism comprise connecting a stimulation device to the pelvic region of the subject, wherein the stimulation device comprises an electrode and the vibration mechanism.

5. The method of claim 4, wherein coupling the stimulation device comprises attaching a cuff at least partially around a scrotum of the subject, wherein the cuff comprises the electrode and the vibration mechanism, wherein the electrode and the vibration mechanism are arranged to engage the scrotum when the cuff is attached to the scrotum.

6. The method of claim 4, comprising positioning the vibration mechanism in a first area of the pelvic region and positioning the electrode in a second area of the pelvic region.

7. The method of claim 1, wherein coupling the electrical energy source comprises adhering an electrode to the subject.

8. The method of claim 7, wherein delivering the electrical energy comprises delivering an electrical pulse at a frequency in a range of approximately 0 Hz to approximately 150 Hz.

9. The method of claim 7, wherein applying vibrations comprises applying vibrations having a frequency in a range of approximately 0 Hz to approximately 50 Hz.

10. The method of claim 1, wherein applying vibrations and delivering the electrical energy comprise delivering an electrical pulse and applying vibrations to a testicular cord, a cremasteric muscle, an inguinal canal, a genitofemoral nerve, an ilioinguinal nerve, and/or an iliohypogastric nerve of the subject.

11. A method of treating testicular pain in a subject, the method comprising:
coupling a stimulation device to a pelvic region of the subject, the stimulation device comprising an electrical contact configured for delivering electrical energy and a vibration mechanism configured for delivering vibrations;
delivering, via the stimulation device, electrical energy to the subject; and
delivering, via the stimulation device, vibrations to the subject.

12. The method of claim 11, wherein coupling the stimulation device comprises engaging one or more of the electrical contacts and the vibration mechanism to a scrotum of the subject.

13. The method of claim 12, wherein coupling the stimulation device comprises positioning a second electrical contact of the stimulation device in an area of the pelvic region that is different than the scrotum of the subject.

14. The method of claim 11, wherein coupling the stimulation device comprises fastening a cuff of the stimulation device around a scrotum of the subject, wherein the electrical contact and the vibration mechanism are integrated with the cuff of the stimulation device.

15. The method of claim 11, wherein delivering electrical energy comprises delivering an electrical pulse to the subject.

16. The method of claim 15, comprising adjusting, via a controller, an intensity of a deliverable electrical energy and/or deliverable vibrations, wherein the controller is operatively coupled to the electrical contact and to the vibration mechanism.

17. The method of claim 16, wherein adjusting comprises adjusting a stimulation parameter using a personal electronic device, wherein the controller is communicatively coupled to the personal electronic device.

18. A wearable device for treating testicular pain, the device comprising:
a first stimulation contact configured for delivering an electrical pulse to a pelvic region of a subject; and
a second stimulation contact configured for delivering vibrations to the pelvic region of the subject.

19. The device of claim 18, comprising a flexible band arranged for removably attaching the first stimulation contact and the second stimulation contact to a scrotum of the subject, wherein the first stimulation contact and second stimulation contact are coupled to the flexible band.

20. The device of claim 18, comprising a controller operatively coupled to the first stimulation contact and the second stimulation contact, the controller comprising a processor, a memory communicatively coupled to the processor and storing executable instructions that, when executed by the processor, causes the processor to:
receive data transmitted by a remote communication device;
send a signal to a generator coupled to the first stimulation contact;
adjust an energy parameter of the electrical pulse generated by the generator;
send a signal to a vibratory element of the second stimulation contact; and
adjust an intensity parameter of vibrations generated by the vibratory element.
